(19) United States
(12) Patent Application Publication
(10) Pub. No.: US 2009/0204118 A1
(43) Pub. Date: Aug. 13, 2009

(54) SURGICAL CABLE WITH MALLEABLE LEADER SEGMENT

(76) Inventor: WILLIAM RALPH PRATT, Newbury Park, CA (US)

Correspondence Address: KOPPEL, PATRICK, HEYBL & DAWSON, 2815 Townsgate Road, SUITE 215, Westlake Village, CA, 91361-5827, US

(21) Appl. No.: 12/369,682

(22) Filed: Feb. 11, 2009

Related U.S. Application Data
(60) Provisional application No. 61/065,724, filed on Feb. 13, 2008.

(51) Int. Cl. A61B 17/68

(52) U.S. Cl. 606/74

(57) ABSTRACT

A surgical cable comprises a core segment, at least one leader segment, and an outer jacket. The core segment is made from a material having a high tensile strength and which is capable of elongation. Each leader segment is arranged axially in tandem with the core segment and comprises a semi-rigid ductile material capable of being manipulated into a desired shape. A plurality of braided fibers form the outer jacket, which surrounds the core segment and at least a portion of the leader segment. The cable is manipulated by means of the leader segments, which are preferably capable of resisting bending in response to head-on compression, thereby enabling the cable to be more easily manipulated around and through anatomical structures.

### RELATED APPLICATIONS

This application claims the benefit of provisional patent application No. 61/065,724, filed Feb. 13, 2008.

### BACKGROUND OF THE INVENTION

1. Field of the Invention

This invention relates generally to surgical cables, and more particularly, to means by which such cables can be more easily manipulated around and through anatomical structures.

2. Description of the Related Art

Many products are known which serve to hold human body tissues and bones in a desired relationship or position, to aid in their healing when injured or diseased. One such product is the surgical cable, which is wrapped around one or more tissues and/or bones as needed. For example, a surgical cable can be wrapped around the fragments of a fractured bone, such that a compressive force is applied which aids in the healing of the bone. Such a cable is described, for example, in U.S. Pat. No. 6,589,246 to Hack et al.

Cables of this sort must be threaded around and through anatomical structures. This requires the cable's leading end to be manipulated by the surgeon, which can be extremely challenging when working in tightly confined spaces, particularly those near highly delicate areas such as the spinal column.

One technique employed to make it easier to manipulate a surgical cable involves swaging a needle onto one or both ends of the cable; one such example is described in U.S. Pat. No. 5,456,722 to McLead et al. The rigidity of the needle simplifies the task of threading it, and its cable, through a confined space. However, this approach can be problematic, especially when employed with a cable that features an inner core encapsulated in a braided outer jacket. To keep the cable components encapsulated within the jacket, the needle would need to be swaged onto the inner core element. Unfortunately, the diameter of the portion of the needle overlapping the core would necessarily be larger than that of the core, thereby complicating the installation of the outer jacket and possibly rendering the cable unsuitable for some applications. A needle might alternatively be swaged onto the cable over the outer jacket; however, this could risk damage to the jacket and unacceptably increase the effective outer diameter of the cable construct.

### SUMMARY OF THE INVENTION

A surgical cable having a malleable leader segment is presented, in which the leader segment facilitates the manipulation of the cable around and through anatomical structures.

The present cable is designed to apply a continuous active compressive force across one or more anatomical structures. The cable includes a core segment, at least one leader segment, and an outer jacket. The core segment is made from a material having a high tensile strength and which is capable of elongation. Each leader segment is arranged axially in tandem with the core segment and comprises a semi-rigid ductile material capable of being manipulated into a desired shape. A plurality of braided fibers form the outer jacket, which surrounds the core segment and at least a portion of the leader segment.

When so arranged, the leader segments enable the cable to be more easily manipulated around and through anatomical structures. The cable is manipulated by means of the leader segments, which are preferably capable of resisting bending in response to head-on compression, at least to the extent needed to push through soft tissues or minor obstructions under manual pressure. Leader segments are preferably located at or near one or both ends of the cable. The core and leader segments are preferably encapsulated within the outer jacket, which is facilitated by ensuring that the leader segment diameter is approximately equal to or less than the diameter of the core segment. If desired, a leader segment can be preformed into a desired shape, such as a J-hook, helix, spiral or eyelet shape.

In one embodiment, the core segment is made from a biocompatible polymer and the leader segments are made from a biocompatible metal. In another embodiment, both the core and leader segments are made from the same semi-rigid ductile material. Yet another embodiment features a core which is a hollow or multi-lumen tube, combined with one or more leader segments made from a semi-rigid ductile material.

These and other features, aspects, and advantages of the present invention will become better understood with reference to the following drawings, description, and claims.

### BRIEF DESCRIPTION OF THE DRAWINGS

FIG. 1 shows perspective views of one possible embodiment of a surgical cable in accordance with the present invention, with one view showing an intact cable and another view showing a cutaway view of the cable.

FIGS. 2a-2c illustrate several possible shapes that a preformed leader segment might take if used with a surgical cable per the present invention.

FIG. 3 is a sectional view of a surgical cable in accordance with the present invention illustrating the use of a leader segment having a composite construct.

FIG. 4a is a flow chart illustrating one possible method by which a surgical cable in accordance with the present invention might be fabricated.

FIG. 4b is a flow chart illustrating another possible method by which a surgical cable in accordance with the present invention might be fabricated.

FIG. 5 is a perspective view of another possible embodiment of a surgical cable in accordance with the present invention.

FIG. 6 is a flow chart illustrating one possible method by which a surgical cable in accordance with the present invention might be manipulated.

### DETAILED DESCRIPTION OF THE INVENTION

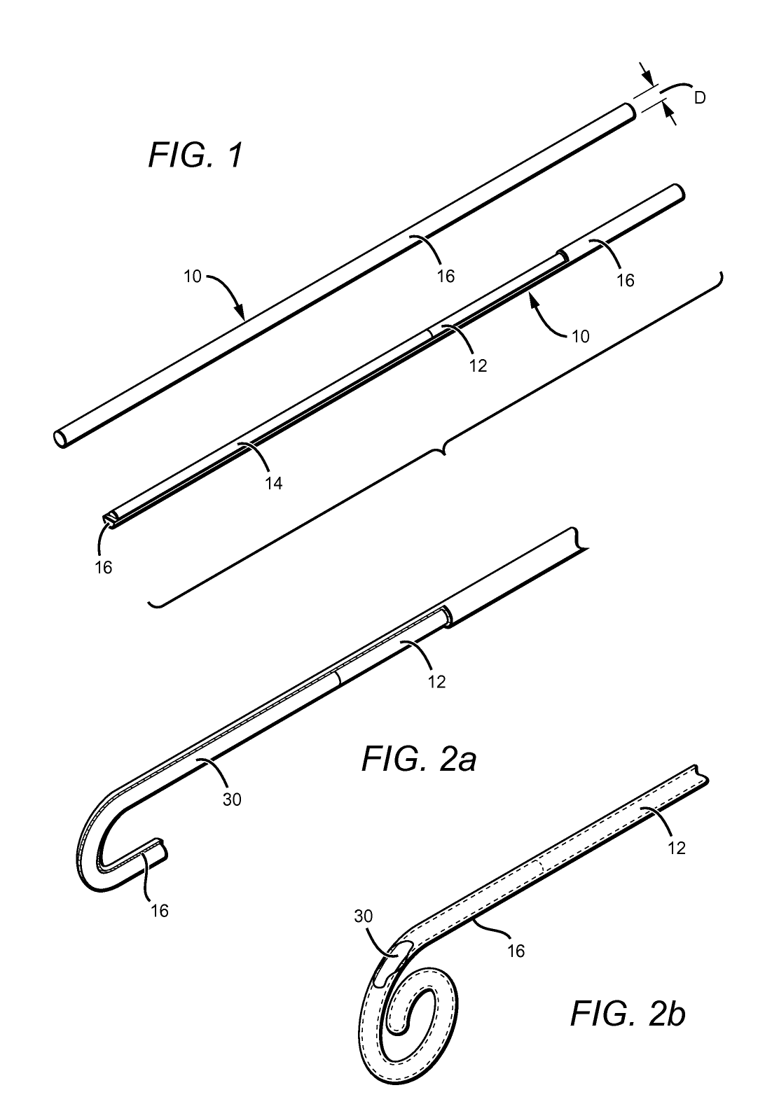

FIG. 1 shows a first embodiment of a surgical cable in accordance with the present invention. Two views are shown: one shows an intact cable 10, and the other shows cable 10 with its outer jacket partially cut away to reveal its core and “leader” segments.

The cable comprises a core segment 12, made from a material having a high tensile strength and capable of elongation, at least one leader segment 14 arranged axially in tandem with core segment 12 and made from a semi-rigid ductile material such that it is malleable—i.e., capable of being manipulated into a desired shape. The cable also includes a plurality of braided fibers that form an outer jacket 16 which surrounds the core segment and at least a portion of leader segment 14. Outer jacket 16 preferably comprises woven or braided fibers made from a high strength, low stretch protective material; a polymer material such as ultra-high molecular weight polyethylene (UHMWP), is preferred. Providing a leader segment made from a semi-rigid ductile material as described herein enables the cable to be more easily manipulated around and through anatomical structures.

The cable is arranged such that at least one leader segment 14 is located at or near one terminus of the cable; typically, two leader segments would be located at respective cable ends. In a preferred embodiment, the core and leader segments are completely encapsulated within outer jacket 16. This has the benefit of maintaining the integrity of the jacket, simplifying the sterilization challenge compared to a swaged-on needle, and eliminating the risk of the leader segment pulling off of the cable or migrating axially out of the jacket during manipulation. This has the additional benefit of creating a smooth transition surface between the leader and core segments of the cable construct, thus preventing an abrupt transition between leader and cable as might be present with a swaged-on needle—which could present a mechanical hazard to adjacent tissue structures as the cable is manipulated past them.

One way in which jacket 16 can be made to encapsulate the core and leader segments is by arranging the jacket such that it can be fused or otherwise bonded at its terminal ends, using heat or adhesive, for example. The leader segments and jacket might also be arranged such that at least a portion of the leader segments can be fused to the jacket. For example, one or more leader segments might be coated or encapsulated with a material capable of being fused with the material with which jacket 16 is made. A leader segment might also be arranged such that it can be fused to its adjacent core segment.

As noted above, leader segments 14 are made from a semi-rigid ductile material capable of being manipulated into a desired shape; biocompatible metals such as steel, titanium, gold, stainless steel, chrome-cobalt alloy or a biocompatible polymer, or a composite thereof, are preferred, for the purpose of patient safety both in terms of blood contact and either intentional or unintentional implantation. In one possible embodiment, the composition and diameter of each leader segment is arranged such that it is plastically deformable and semi-rigid when at or near body temperature, in response to forces applied manually across its longitudinal axis either by hand or with the aid of instruments. Such a construction results in a low-profile, minimally invasive leader that aids in inserting or threading the cable in, around, and behind tissue structures—such as bone—and through highly confined spaces with a minimal risk of damage to adjacent critical and delicate tissue structures such as arteries and nerves. The malleable leader segment benefits the surgeon by being readily shaped into a multitudinous range of configurations that the surgeon may find necessary for successful passage of the cable, thus providing the surgeon with intra-operative flexibility when directing the cable through confined spaces, such as those found along the spinal column. For example, a surgeon can conveniently form the leader segment into a “J” shape for hooking around a bony process without the necessity of using a bulky tubular instrument to facilitate and direct passage of the cable.

The mechanical demands of a particular surgical application and the need for a sterilizable, biocompatible material should be considered when selecting the leader segment material. The diameter, metallurgical state, and composition of the leader segment should also be chosen to provide a balance between rigidity and plastic-deformability (ductility); the leader segment is preferably made rigid enough to prevent being easily turned aside or bent by end-on encounters with resilient obstructions such as soft tissues or fat. In one suitable embodiment, the cable's leader segment comprised a titanium wire with a diameter of 0.032 inches and its outer jacket was suitably 0.062 inches in diameter and comprised of woven fibers of UHMWP material. The length of the leader segment may be typically in the range of 1 to 4 inches; these measurements are only by way of example. Leader segment specimens having a diameter as small as 0.025 inches and as large as 0.040 inches have been produced.

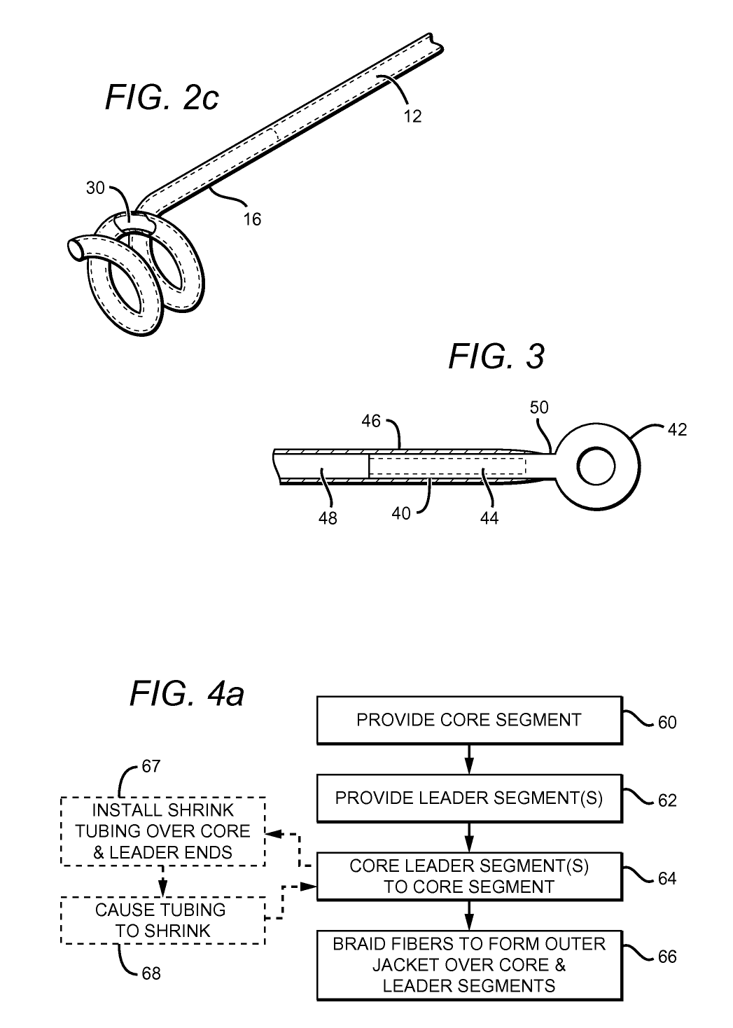

Alternatively, the leader segment need not be easily plastically deformable with low force and at or near body temperatures, as industrial forming techniques could be employed to preform the leader segment into a desired shape. For example, as shown in FIGS. 2a-2c, a leader segment 30 could be advantageously preformed into a J-hook, helix or spiral shape, respectively, with a material with rigidity appropriate to the specific application.

In another possible embodiment, the leader segment can include specialized end forms and extensions which might be required for surgical advantage, which could be fused to the outer jacket and/or to the core segment. For example, the leader segment itself could be a composite construct consisting of a malleable or suitably rigid wireform encased in a material of a fusable nature with the material of the jacket. For example, in FIG. 3, the leader segment 40 comprises an eyelet 42 made from molded plastic, which has a core 44 made from a semi-rigid ductile core material. The cable's outer jacket 46 encases the cable's core segment 48 and a portion of leader segment 40, and is preferably fused to the leader segment at the base 50 of the round portion of the eyelet. Note that an eyelet is but one possible example of a leader segment of this sort; a leader segment made from a fusable material with a semi-rigid ductile core could be formed into virtually any desired shape.

In one embodiment, the cable's core segment comprises a biocompatible polymer, such as nylon, polyester, polyethylene, fluorocarbon or polyetheretherketone (PEEK); at least one filament of a relatively low modulus polymer capable of high elongation (such as nylon monofilament) is preferred. Additional details concerning a cable of this type can be found in U.S. Pat. No. 6,589,246 to Hack et al. The core segment would typically run most of the working length of the cable, with relatively short leader segments at one or both ends.

The core segment of a cable as described above has a solid cross-section. Alternatively, the core segment can comprise a hollow or multi-lumen tube, such as a catheter. In this case, one or more semi-rigid ductile leader segments would be arranged axially in tandem with the tube, and both the tube and leader segments would be contained within an outer jacket as described above. When so arranged, the leader segments can be manipulated as needed to install the tube in a desired location.

The present surgical cable can be made such that each leader segment abuts a terminus of the core segment, with the outer jacket used to keep the core and leader segments aligned axially. Alternatively, the leader segments can be mechanically coupled to the core segment. One possible way of accomplishing this is discussed below.

The diameter of the leader segments is approximately equal to or less than the diameter of the core segment. Configuring the cable in this way has the benefit of not necessitating the enlargement of the outside diameter of the cable construct, thus maintaining compatibility with existing ancillary instruments and implants such as tensioners and clasping mechanisms. For these reasons, the outside diameter D of the cable preferably does not significantly flare outward near the cable ends.

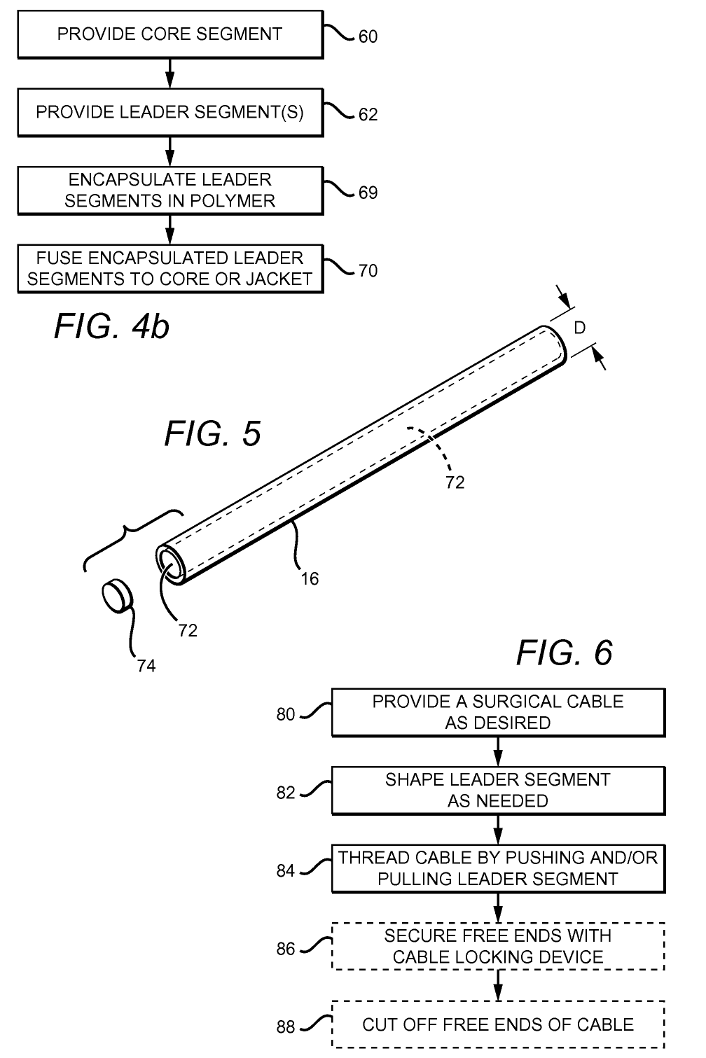

There are a number of ways in which a surgical cable as described above could be fabricated. One possible fabrication method, illustrated in FIG. 4a, proceeds as follows:
provide a core segment made from a biocompatible material having a high tensile strength and capable of elongation (step 60);
provide at least one leader segment comprising a semi-rigid ductile material capable of being manipulated into a desired shape (62);
couple the leader segment to a terminus of the core segment such that the leader segment is arranged axially in tandem with the core segment (64); and
braid a plurality of fibers so as to form an outer jacket which surrounds the core and leader segment (66).

One way in which the core and leader segment can be coupled together is with the use of shrink tubing, which would be installed over a terminus of the leader segment and the terminus of the core segment with which it is in tandem (67). Once installed, the tubing is caused to shrink (68), thereby coupling the leader and core segments together.

Another possible fabrication method is illustrated in FIG. 4b. A core segment (60) and leader segments (62) are provided as described above. Then, the leader segments are encapsulated in a polymer that is fusible to either the core segment or the outer jacket (69), and then fused as appropriate to complete the cable (70).

In another possible embodiment, there is no distinct leader segment; rather, the cable's core segment is made from a semi-rigid ductile material capable of being manipulated into a desired shape and which runs the full length of the cable. A surgical cable of this sort would also include an outer jacket as described above. The core segment preferably comprises biocompatible metals such as steel, titanium alloy, chrome-cobalt alloy, gold or stainless steel, or a biocompatible polymer, or composite thereof. As with the leader segments described above, a portion of the core segment at or near a terminus of the cable could be preformed into a desired shape.

One possible embodiment of a surgical cable of this type is shown in FIG. 5, which depicts a cable sliced and exploded to reveal a cross-section. Here, the cable's core 72 comprises a semi-rigid, ductile material over the cable's entire working length. The end of the cable (74) would normally be integrated with the length of cable, but preferably fused or otherwise sealed to contain the core 72. While this species may lack the capability for a high degree of elongation (in comparison with an embodiment featuring a polymer core as described above), it has independent advantages and is suited to certain surgical applications. For example, semi-rigid, ductile core 72 can possess rigidity sufficient to resist bending when met with axial compression forces within a range sufficient to permit the cable to be thrust manually forward, causing it to penetrate through minor anatomical obstructions such as soft tissue or fatty tissues. Assuming the core comprises metal, it also imparts tensile strength, with high modulus.

As noted above, a surgical cable of this sort includes a braided polymer outer jacket, which allows a cable to be readily used in contact with metallic implants without direct metal-to-metal contact. This reduces the potential for wear debris and galvanic corrosion, and if breakage should occur, metallic fragments in the core cable are contained by the outer jacket, rather than being released into the body. The outer jacket, preferably formed of UHMWP, also is resistant to abrasion and slides easily across surfaces without catching. The jacket also tends to prevent kinking by maintaining a minimum radius at bent corners; the absence of abrupt kinks tends to prevent breaking under tension or fatigue loading.

A cable of this sort, with a continuous semi-rigid core, can be manufactured by simply braiding the polymer outer jacket around a tensed metallic core, with conventional machinery. It should be understood that both the semi-rigid and polymer core cables are preferably tested, sterilized, and packaged to maintain sterility during distribution.

One method by which a surgical cable as described herein would be manipulated, illustrated in FIG. 6, is as follows:
a surgical cable as described herein is provided (step 80);
the leader segment is shaped so as to enable the cable to be more easily manipulated around and through particular anatomical structures (82); and
the leader segment is pushed and/or pulled by the surgeon as needed to thread the cable around and through the anatomical structures (84).
   The leader segment may be shaped by either bending it as needed during a surgical procedure, or preforming it into a desired shape prior to the commencement of a surgical procedure in which the cable is employed.

Once the cable has been threaded as needed, its free ends may be secured with, for example, a cable locking device (86). The cable locking device is preferably arranged such that, once the cable is threaded as desired, the free ends of the threaded cable extend from the device; the free ends are then cut off approximately flush with the cable locking device (88). One suitable cable locking device is described in U.S. Pat. No. 7,207,090 to Mattchen.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

I claim:

1. A surgical cable for applying a continuous active compressive force across one or more anatomical structures, comprising:
a core segment made from a material having a high tensile strength and capable of elongation;
at least one leader segment arranged axially in tandem with said core segment, said at least one leader segment comprising a semi-rigid ductile material capable of being manipulated into a desired shape; and
a plurality of braided fibers forming an outer jacket which surrounds said core segment and at least a portion of said at least one leader segment;
said at least one leader segment enabling said cable to be more easily manipulated around and through anatomical structures.

2. The cable of claim 1, wherein at least one of said leader segments is located at or near one terminus of said cable.

3. The cable of claim 1, wherein two of said leader segments are located at respective ends of said cable.

4. The cable of claim 1, wherein said core segment and said at least one leader segment are encapsulated within said jacket.

5. The cable of claim 4, wherein said jacket is fused at its terminal ends.

6. The cable of claim 1, wherein said at least one leader segment is fused with said jacket and /or said core segment.

7. The cable of claim 6, wherein said at least one leader segment is coated or encapsulated with a material capable of being fused with said jacket and/or said core segment.

8. The cable of claim 1, wherein said leader segments comprise biocompatible metals, a biocompatible polymer, or a composite thereof.

9. The cable of claim 8, wherein said biocompatible metals comprise steel, titanium, gold, chrome-cobalt alloy or stainless steel.

10. The cable of claim 1, wherein said at least one leader segment abuts a terminus of said core segment.

11. The cable of claim 1, wherein said at least one leader segment is coupled to a terminus of said core segment.

12. The cable of claim 11, further comprising shrink tubing arranged to couple said at least one leader segment to said core segment.

13. The cable of claim 1, wherein the diameter of said at least one leader segment is approximately equal to or less than the diameter of said core segment.

14. The cable of claim 1, wherein the composition and diameter of said at least one leader segment are arranged such that said segment is plastically deformable in response to forces applied manually across its longitudinal axis.

15. The cable of claim 1, wherein said at least one leader segment is preformed into a desired shape.

16. The cable of claim 15, wherein said at least one leader segment is preformed into a J-hook, helix, spiral or eyelet shape.

17. The cable of claim 1, wherein said at least one leader segment further comprises a preformed component, at least a portion of which is made from a fusable material, said component encasing at least a portion of said at least one leader segment's semi-rigid ductile material, said cable arranged such that said component's fusable material is fused to said jacket and /or said core segment.

18. The cable of claim 1, wherein said core segment comprises a biocompatible polymer.

19. The cable of claim 18, wherein said core segment comprises nylon, polyester, polyethylene, fluorocarbon or polyetheretherketone (PEEK).

20. The cable of claim 1, wherein said core segment comprises said semi-rigid ductile material capable of being manipulated into a desired shape, said core segment and said at least one leader segment being continuous.

21. The cable of claim 1, wherein said core segment comprises a hollow or multi-lumen tube.

22. The cable of claim 21, wherein said core segment is a continuously hollow tube, wherein two of said leader segments are located at respective ends of said tube.

23. The cable of claim 1, wherein said plurality of braided fibers comprise a high strength, low stretch protective material.

24. The cable of claim 23, wherein said plurality of braided fibers comprise ultra-high molecular weight polyethylene (UHMWP).

25. The cable of claim 1, wherein each of said cable's constituent components are biocompatible and sterilizable.

26. A surgical cable for applying a continuous active compressive force across one or more anatomical structures, comprising:
a core segment made from a semi-rigid ductile material capable of being manipulated into a desired shape; and
a plurality of braided fibers forming an outer jacket which surrounds said core segment, said plurality of braided fibers comprising a high strength, low stretch protective material;
said semi-rigid ductile material enabling said cable to be more easily manipulated around and through anatomical structures.

27. The cable of claim 26, wherein said core segment comprises biocompatible metals, a polymer, or a composite thereof.

28. The cable of claim 27, wherein said biocompatible metals comprise steel, titanium alloy, chrome-cobalt alloy, gold or stainless steel.

29. The cable of claim 26, wherein a portion of said core segment at or near a terminus of said cable is preformed into a desired shape.

30. A method of fabricating a surgical cable for applying a continuous active compressive force across one or more anatomical structures, comprising:
providing a core segment made from a biocompatible material having a high tensile strength and capable of elongation;
providing at least one leader segment comprising a semi-rigid ductile material capable of being manipulated into a desired shape;
coupling said at least one leader segment to a terminus of said core segment such that said leader segment is arranged axially in tandem with said core segment; and
braiding a plurality of fibers so as to form an outer jacket which surrounds said core segment and said at least one leader segment.

31. The method of claim 30, wherein said coupling comprises:
installing shrink tubing over a terminus of said at least one leader segment and said terminus of said core segment; and
causing said tubing to shrink.

32. The method of claim 30, further comprising encapsulating said at least one leader segment in a polymer that is fusible to said core segment;
wherein said coupling comprises fusing said encapsulated leader segments to said core segment.

33. A method of fabricating a surgical cable for applying a continuous active compressive force across one or more anatomical structures, comprising:
providing a core segment made from a biocompatible material having a high tensile strength and capable of elongation;
providing at least one leader segment comprising a semi-rigid ductile material capable of being manipulated into a desired shape;
encapsulating said at least one leader segment in a fusible polymer;
braiding a plurality of fibers so as to form an outer jacket which surrounds said core segment and said at least one leader segment; and
fusing said encapsulated leader segments to said outer jacket.

34. A method of manipulating surgical cable around and through anatomical structures, comprising:
providing a surgical cable comprising:
a core segment having a high tensile strength and capable of elongation;
at least one leader segment comprising a semi-rigid ductile material capable of being manipulated into a desired shape; and
a plurality of braided fibers forming an outer jacket which surrounds said core segment and said at least one leader segment;
bending said at least one leader segment into a shape which enables said cable to be more easily manipulated around and through particular anatomical structures; and
using said at least one bent leader segment to thread said cable around and through said anatomical structures.

35. The method of claim 34, wherein said at least one leader segment is bent as needed during a surgical procedure in which said cable is employed.

36. The method of claim 34, wherein said at least one leader segment is preformed into a desired shape prior to the commencement of a surgical procedure in which said cable is employed.

37. The method of claim 34, further comprising securing the free ends of said threaded cable with a cable locking device.

38. The method of claim 37, wherein said cable locking device is arranged such that the free ends of said threaded cable extend from said cable locking device, further comprising cutting off said free ends approximately flush with said cable locking device.
